(19) United States
(12) Patent Application Publication
(10) Pub. No.: US 2008/0177280 A1
(43) Pub. Date: Jul. 24, 2008

(54) Method for Depositing Radiation in Heart Muscle

(75) Inventors: John Adler, Stanford, CA (US); Thilaka Sumanaweera, Los Altos, CA (US); Patrick Maguire, Menlo Park, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: CYBERHEART, INC., Menlo Park, CA (US)

(21) Appl. No.: 11/971,725

(22) Filed: Jan. 9, 2008

Related U.S. Application Data
(60) Provisional application No. 60/879,724, filed on Jan. 9, 2007.
(60) Provisional application No. 60/879,654, filed on Jan. 9, 2007.

(51) Int. Cl. A61B 19/00

(52) U.S. Cl. 606/130; 901/41

(57) ABSTRACT

Radiosurgical treatment of tissues of the heart to mitigate arrhythmias such as atrial fibrillation or the like. Radiosurgical targeting of the relatively rapid movement of heart tissues may be enhanced by generating a moving model volume using a time-sequence of three dimensional acquired tissue volumes. A digitally reconstructed radiograph (DRR) may be generated from the model at a desired cardiac and/or respiration motion phase and compared to an X-ray or the like taken immediately before or during treatment. When a series of radiation beams will be directed to a heart tissue to alleviate an arrhythmia, the treatment system may alter the radiation beam series in response to the type of the arrhythmia.

### CROSS-REFERENCES TO RELATED APPLICATIONS

This application claims the benefit of under 35 U.S.C. §109(e) of U.S. Provisional Patent Application Nos. 60/879,724 and 60/879,654; both filed on Jan. 9, 2007, the disclosures of which are incorporated herein by reference.

### BACKGROUND OF THE INVENTION

The present invention generally provides improved methods, devices, and systems for treatment of tissue, in many cases by directing radiation from outside the body toward an internal target tissue. Exemplary embodiments may deposit a specified radiation dose at a moving target tissue such as a target in the heart muscle while limiting or minimizing the dose received by adjoining and/or critical tissue structures.

In the past, targets such as tumors in the head, spine, abdomen and lungs have been successfully treated by using radiosurgery. During radiosurgery, the target is bombarded with a series of beams of ionizing radiation (for example, a series of MeV X-ray beams) fired from various different positions and orientations by a radiation delivery system. The beams can be directed through intermediate tissue toward the target tissue so as to affect the tumor biology. The beam trajectories help limit the radiation exposure to the intermediate and other collateral tissues, using the cumulative radiation dose at the target to treat the tumor. The CyberKnife™ Radiosurgical System (Accuray Inc.) and the Trilogy™ radiosurgical system (Varian Medical Systems) are two such radiation delivery systems.

Modern robotic radiosurgical systems may incorporate imaging into the treatment system so as to verify the position of the target tissue without having to rely on rigid frameworks affixing the patient to a patient support. Some systems also have an ability to treat tissues that move during respiration, and this has significantly broadened the number of patients that can benefit from radiosurgery. It has also previously been proposed to make use of radiosurgical treatments for treatment of other tissues that undergo physiological movements, including the directing of radiation toward selected areas of the heart for treatment of atrial fibrillation.

During atrial fibrillation, the atria lose their organized pumping action. In normal sinus rhythm, the atria contract, the valves open, and blood fills the ventricles (the lower chambers). The ventricles then contract to complete the organized cycle of each heart beat. Atrial fibrillation has been characterized as a storm of electrical energy that travels across the atria, causing these upper chambers of the heart to quiver or fibrillate. During atrial fibrillation, the blood is not able to empty efficiently from the atria into the ventricles with each heart beat. By directing ionizing radiation toward the heart based on lesion patterns used in open surgical atrial fibrillation therapies (such as the Maze procedure), the resulting scar tissue may prevent recirculating electrical signals and thereby diminish or eliminate the atrial fibrillation.

While the proposed radiosurgical treatments of atrial fibrillation offer benefits by significantly reducing trauma for heart patients, improvements to existing radiosurgical systems may be helpful to expand the use of such therapies. For example, movement of the tissues of the heart during a heartbeat may be significantly more rapid than movements of lung tumors induced by respiration. While well suited for treatment of lung tissues and the like, existing systems used to verify target registration may also limit radiation exposure of collateral tissues and/or avoid delays in the procedure by limiting the rate at which x-ray images are acquired during treatment. As several radiation-sensitive structures are in and/or near the heart, and as the treatment time for a single heart patient may be as long as 30 minutes or more, increasing the imaging rate and/or delaying the radiation beams when the target tissue is not sufficiently aligned may be undesirable in many cases.

In light of the above, it would be desirable to provide improved devices, systems, and methods for treating moving tissues of a patient, particularly by directing radiation from outside the patient and into target tissues of a heart. It would be particularly beneficial if these improvements were compatible with (and could be implemented by modification of) existing radiosurgical systems, ideally without significantly increasing the exposure of patients to incidental imaging radiation, without increasing the costs so much as to make these treatments unavailable to many patients, without unnecessarily degrading the accuracy of the treatments, and/or without causing collateral damage to the healthy tissue despite the movement of the target tissues during beating of the heart.

### SUMMARY OF THE INVENTION

The present invention generally provides improved medical devices, systems, and methods, particularly for treatment of moving tissues. The invention allows improved radiosurgical treatment of tissues of the heart, often enhancing the capabilities of existing robotic radiosurgical systems for targeting tissues of the heart to mitigate arrhythmias such as atrial fibrillation or the like. Radiosurgical targeting of the relatively rapid movement of heart tissues may be enhanced by generating a moving model volume using a time-sequence of three dimensional (3-D) acquired tissue volumes. These acquired tissue volumes may be obtained using computed tomography (CT), magnetic resonance imaging (MRI), positron emission tomography (PET), ultrasound, or the like. Associated with each of the 3-D tissue volumes, cardiac cycle data will also be included in the model volume, such as by obtaining electrocardiogram (ECG or EKG) measurements during acquisition of the tissue volumes. Optionally, the motion model may be separated into two components, with the first portion of the model comprising a cardiac motion model and the second portion of the model comprising a respiration motion model. A digitally reconstructed radiograph (DRR) may be generated from the model at a desired cardiac and/or respiration motion phase. The DRR can then be compared to a planar image such as an X-ray or the like taken immediately before or during treatment. In some embodiments, a separate intra-operative motion model may be generated by acquiring a time sequence of images using bi-plane X-ray imaging capabilities of the treatment system. It may be advantageous to image surface fiducials (such as light-emitting diodes (LEDs) mounted to the skin of the patient using standard surface imaging cameras to determine respiration-induced movement of a target tissue using the intra-operative model, often while monitoring heart cycle signals (such as an ECG signal) for determining heartbeat-induced motion of the target tissue (also using the intra-operative model). When directed to a heart tissue to alleviate an arrhythmia, the treatment system may alter the radiation beam series in response to the type of the arrhythmia.

In a first aspect, the invention provides a method for treating a moving target tissue. The method comprises acquiring at least one image of the target tissue and generating a simulated image from a model volume. A similarity measure is computed between the image or images and the simulated image. A robot is configured in response to the similarity measure, and a radiation beam is fired from the configured robot.

The target tissue will often comprise a target heart tissue within a heart of the patient. A series of radiation beams can be fired from the robot along different trajectories from outside the patient. The model volume may be generated before the series of radiation beams by acquiring a time-sequence of volumes (optionally using CT scans), and associated cardiac cycle phase measurements (such as ECG data). The model volume will typically comprise a model of movement of the target tissue correlated to the cardiac phase signals. The simulated image will often have an associated cardiac phase. In some embodiments, the model may be generated by acquiring a time-sequence of volumes and associated respiratory cycle phase measurements or signals. Such embodiments may provide a model of movement of the target tissue (and optionally collateral and sensitive tissues) correlated to the respiratory phase.

Each acquired image may be acquired immediately before and/or during the series of radiation beams, typically between individual beams of the series. The cardiac phase associated with each acquired image may also be identified, typically from cardiac signals acquired using a cardiac sensor. The cardiac phases associated with the simulated image or images and the cardiac phase of the acquired image may be correlated when the similarity measure is computed.

Each volume used in the model volume may be acquired by imaging a plurality of cross-sectional slices across the heart. The target tissue will often be sufficiently limited in contrast within the model volume to inhibit modeling of the target tissue movement throughout the time sequence, and/or to inhibit tracking of the target tissue movement in response to the acquired image. Movement modeling and/or target tracking may be enhanced by temporarily introducing at least one imagable material into the blood within the heart. For example, a contrast agent may be released into the blood to flow into the heart during the time sequence of CT volume scanning. In some embodiments, a catheter may be advanced through a blood vessel and into the heart so as to provide a temporary fiducial within the heart during CT scans and/or X-ray imaging. Regardless, the imagable material need not remain within the heart after treatment. In some embodiments, some or all of the target tissue of the heart may not be visible in the acquired volumes of the model volume and/or in the X-ray images.

Model volume will often comprise a movement model, sometimes referred to as a four dimensional (4-D) model that encompasses the target tissue. Along with the standard three dimensional tissue coordinates, the model may include movement of the tissue with time during a respiration cycle, a cardiac cycle, and/or the like. The movement model may be separated into components, such as a cardiac cycle movement model and a respiration cycle movement model. For example, a time sequence of volumes may be acquired while the patient is holding their breath so as to inhibit respiration-induced movement artifacts. The cardiac cycle movement artifacts may be minimized by selectively obtaining the volumes throughout a respiration cycle, but at a common phase of the cardiac cycle (such as the quiescent T-wave portion of the ECG cycle).

The series of radiation beams may be planned using the model volume, with the motion used to identify the exposure of collateral tissues to differing doses of radiation induced by periodic movement and the like. In many embodiments, the model volume will comprise a pre-treatment model, with an additional intra-operative motion model used during treatment of the target tissues. The intra-operative motion model may be generated by acquiring a time sequence of images from adjacent the target tissue, along with images of external fiducials (such as LEDs mounted to the skin of the patient) throughout the respiration cycle once the patient is positioned for the series of radiation beams. The external fiducials can then be imaged during the series of radiation beams along with monitoring of ECG signals. The motion of the target tissue can be predicted during the series of radiation beams in response to both the imaged external fiducials and the electrocardiogram monitoring. The intra-operative motion model may be intermittently verified by acquiring images from an area adjacent to the target tissue (often using X-ray or the like), with the intermittent images being acquired at a rate that is significantly lower than the respiration rate (and hence much lower than the cardiac cycle rate). This use of external fiducials, intermittent imaging, and the motion model of the model volume allows accurate targeting of the rapidly moving tissues of the heart without subjecting the patient to excessive quantities of radiation through continuous fluoroscopic imaging throughout treatments. Alternative embodiments may employ fluoroscopy imaging, optionally continuously throughout at least a significant portion (or even all) of a treatment.

In another aspect, the invention provides a method for treating a moving target tissue of the heart. The method comprises acquiring at least one computed tomography (CT) volume of the heart. At least one X-ray image of the heart is also acquired, and a digitally reconstructed radiograph (DRR) is generated from the CT volume. A similarity measure is computed between the X-ray and the DRR. A robot is configured dependent on the similarity measure, and a radiation beam is fired from the configured robot.

In another aspect, the invention provides a system for treating a moving target tissue. The system comprises an image acquisition system for acquiring at least one image of the target tissue. A processor is coupled to the image acquisition system. The processor is configured for generating a simulated image from a model volume. The model volume includes a motion model of the target tissue. A similarity measure is computed between the image and the simulated image. A configuration is determined in response to the similarity measure, and a robot coupled to the processor implements the configuration. The radiation beam source is supported by the robot.

An electrogram measurement system may be coupled to the processor. The processor may superimpose the electrogram onto the model volume, and may plan a series of radiation beams so as to inhibit an arrhythmia of the heart using the superimposed electrogram/volume, with the radiation beams typically inhibiting one or more contractile pathways or arrhythmogenic site of the heart.

The targeting of the radiation beams (and hence the configuration of the robot) may be determined by the processor in response to a type of the arrhythmia. In some embodiments, the series of radiation beams may be altered in response to an arrhythmia type signal. For example, where the arrhythmia type signal corresponds to an intermittent arrhythmia, the processor may be configured to interrupt a series of radiation beams when cardiac signals from the sensor indicate an acute arrhythmia event. This may, for example, allow normal cardiac cycle tracking to be employed and temporarily interrupted when an intermittent irregular heartbeat is detected. Other arrhythmia type signals may be treated quite differently. For example, where the arrhythmia type comprises a chronic atrial fibrillation, the processor may interrupt the series of radiation beams if the cardiac signals from an ECG sensor or the like indicate a normal sinus rhythm. This may allow the system to avoid misalignment while taking advantage of limited target movement, optionally with no cardiac cycle adjustments during the arrhythmia.

### BRIEF DESCRIPTION OF THE DRAWINGS

FIG. 1 is an exemplary CyberKnife stereotactic radiosurgery system for use in embodiments of the invention.

FIG. 1A is a graph showing exemplary data from the anterior/posterior motion of a point at the cavotricuspid isthmus inside the right atrium of a pig heart.

FIG. 2 is a graph similar to FIG. 1A showing timing for acquiring a time-sequence of 11 X-ray image pairs over 1 respiratory cycle at a common cardiac phase, Φ.

FIG. 3 is an illustration of an EKG waveform showing exemplary phases where a time-sequence of CT volumes are acquired.

FIG. 4 is an illustration of M×N X-rays, LED signals and ECG signals as acquired over 1 respiratory cycle for use, for example, in an intra-operative motion prediction and validation model.

FIG. 5 schematically illustrates a method for treating a target tissue using a radiosurgical system.

FIG. 5A illustrates a refined method based on that of FIG. 5, in which a moving target tissue of the heart is treated using a radiosurgical system that measures heart cycle signals during imaging and treatment.

FIG. 6 schematically illustrates a more detailed functional block diagram of an exemplary treatment system according to an embodiment of the invention.

### DETAILED DESCRIPTION OF THE INVENTION

The present invention generally provides improved devices, systems, and methods for treatment of tissue, often using radiosurgical systems. The invention is particularly well suited for tracking of moving tissues such as tissues of the heart and tissue structures adjacent the heart that move with the cardiac or heartbeat cycles. The invention may take advantage of structures and methods which have been developed for treating tumors, particularly those which are associated with treatments of tissue structures that move with the respiration cycle. The cardiac cycle is typically considerably faster than the respiration cycle. The overall treatment times can also be quite lengthy for effective radiosurgical procedures on the heart (typically being greater than 10 minutes, often being greater than ½ hour, and in many cases, being two hours or more). Hence, it will often be advantageous to avoid continuous imaging of the target and adjacent tissues using fluoroscopy or the like. Embodiments of the invention may make use of a motion model of a tissue volume encompassing the target tissue. The motion model may be correlated to a heart signal sensor such as an electrocardiogram (ECG) or (EKG). The motion model may be derived by acquiring 3-D volumes while measuring the heart cycle signals, and the heart cycle signals may also be monitored during treatment so as to predict the position of the target tissue. Multiple models may be employed, including separation of the motion model into a cardiac cycle model and a respiration cycle model. In other embodiments, the motion model may be correlated to both cardiac and respiratory cycles. In some embodiments, a pre-treatment model may be used for planning and registration. An intra-operative model may be employed to track motion of the heart during treatment, often in response to external fiducials and/or a heart cycle signal. A variety of differing embodiments may be employed, with the following description presenting exemplary embodiments that do not necessarily limit the scope of the invention.

The present invention may take advantage of many components included in or derived from known radiation delivery system components. Suitable system components may comprise:
1. A linear accelerator (Linac) capable of generating a series of X-ray beams;
2. A mechanism to position and orient the linear accelerator (and, hence, the X-ray beams);
3. A patient registration system to position and orient the target in the coordinate system of the delivery system;
4. A tracking system for tracking the target during treatment in case the target changes shape or moves between the time of, for example, an initial tracking X-ray of a pre-treatment computed tomography (CT) exam and the time of treatment, and/or during treatment due to respiration, patient-induced gross anatomical movement, and the like;
5. A couch capable of positioning the target (patient) independent of the mechanism described in #2 above.

In exemplary CyberKnife-based systems, the above 5 items may correspond to:
A 6 MeV X-band X-ray Linac
A 6 degree-of-freedom (DOF) robotic manipulator.
A patient registration system consisting of:
   Two ceiling-mounted diagnostic X-ray sources
   Two amorphous silicon image detectors mounted on the floor.
During treatment, two orthogonal X-rays are taken and registered with the CT data by cross-correlating the X-rays with simulated X-rays generated by CT data, called digitally reconstructed radiographs (DRR).
The tracking system may include several light-emitting diodes (LEDs) mounted on the patent's skin to provide additional information at a rate faster than what X-rays alone provide.
A couch with 5 DOF.

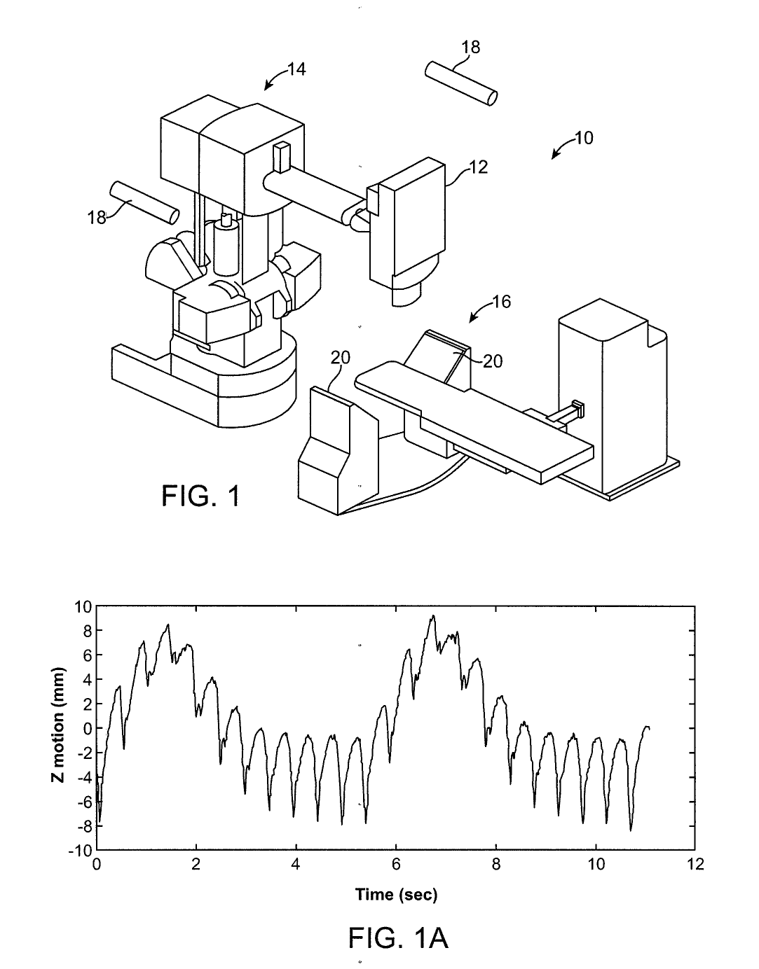

An exemplary CyberKnife stereotactic radiosurgery system 10 is illustrated in FIG. 1.

Radiosurgery system 10 has a single source of radiation, which moves about relative to a patient. Radiosurgery system 10 includes a lightweight linear accelerator 12 mounted to a highly maneuverable robotic arm 14. An image guidance system 16 uses image registration techniques to determine the treatment site coordinates with respect to linear accelerator 12, and transmits the target coordinates to robot arm 14 which then directs a radiation beam to the treatment site. When the target moves, system 10 detects the change and corrects the beam. Hence, system 10 makes use of robot arm 14 and linear accelerator 12 under computer control. Image guidance system 16 includes diagnostic x-ray sources 18 and image detectors 20, this imaging hardware comprising two diagnostics fluoroscopes. These fluoroscopes provide a frame of reference for locating the patient's anatomy, which, in turn, has a known relationship to the reference frame of robot arm 14 and linear accelerator 12.

Pre-Treatment Imaging and Treatment Planning

Typically, the target and its surrounding tissue are first imaged using CT, resulting in a volume of data. The target volume is then delineated in this CT volume and a desired dose to the target is prescribed. Delicate or other tissue structures of concern in the vicinity of the target are also delineated and may be assigned a maximum desired dose that can be deposited at these structures. A computer program then receives the location and the shape of the target and the critical structures, the prescribed doses and the geometric configuration of the radiation delivery system and computes (a) the position and orientation of the beams to be fired and (b) a contour diagram showing dose received by all voxels in the CT volume. The radiation oncologist then reviews this data to see if the target is receiving the right dose and if structures in the vicinity receive too much dose. He or she may modify the boundaries of the target and the critical structures, along with dose received by them, to reach an acceptable treatment plan.

Treatment Delivery

During treatment delivery, the target can be first registered with the coordinate system of the treatment delivery system by using the patient registration system. The treatment delivery system may also receive the beam positions and orientations from the treatment planning stage. It then positions and orients the Linac and fires the beams towards the target.

Treatment Delivery in the Presence of Respiratory Motion

A preferred robot manipulator may be capable of positioning and orienting the Linac so that it follows the target due to breathing in real time. Since Fluoroscopic imaging may be disadvantageous for the entire duration of the radiation delivery (optionally about 2 hours or more) because it subjects the patient to extra radiation, the tracking system may first build an intra-operative correlation model between the motion of the skin of the patient recorded by the imaging of external light emitting diodes (LEDs) mounted to the skin of the patient and any fiducials implanted in the vicinity of the target and seen in the X-rays. (The tumor itself need not be visible in the X-rays). Tracking of the LEDs using one or more cameras oriented toward the skin of the patient can then be used to determine data regarding the respiration cycle and the positions of tissues that move with the respiration cycle. More specifically, intra-operative correlation models can be built by taking a series of X-ray images in quick succession for one or more breathing cycles and at the same time, recording the position of the skin using the signals from the LEDs. Following this, the LED signals alone may be used for at least a portion of the tracking. X-rays may be intermittently acquired to verify the validity of the correlation model. If the model is no longer sufficiently valid, a fresh model is generated by following the same procedure as before.

Targets in the heart (tumors or other types of targets) poses two challenges for radiation delivery systems:
Implantation of fiducials in the heart muscle can be difficult and/or disadvantageous.
The heart itself beats fairly rapidly (for example, roughly at a rate of 1 beat every second), and some parts of the heart move more than the other parts due to this beating. In addition, the heart as a whole may also move due to respiration.

FIG. 1A graphically shows the anterior/posterior motion of a point at the cavotricuspid isthmus inside the right atrium of a pig heart. As can be seen, the motion has two components: a slow varying breathing component and a rapidly varying cardiac component.

Embodiments of the present invention address either and/or both the above challenges and facilitates radiosurgery of targets in the heart muscle. Optionally, a beam of radiation may be redirected in response to a model including the target tissue, and/or a beam of radiation may be gated in response to the model.

Case 1: No (or Negligible) Cardiac Component; with Significant Respiratory Component

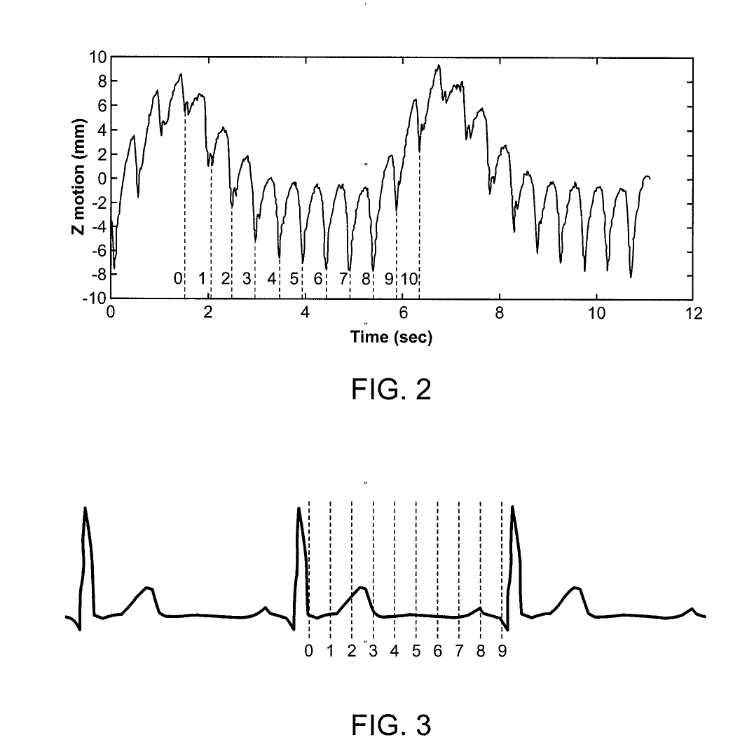

In this case, the target in the heart muscle has only a respiratory component and not a cardiac component. Targets in the left atrium near the pulmonary veins may fall into this category. The steps may include:
1. Acquire a single CT volume at a cardiac phase, Φ, of the cardiac cycle. Use a high speed CT scanner such as the 64-slice Siemens SOMOTOM Definition to acquire CT volumes quickly, e.g. one volume in 83 ms. Contrast agents may be used. Outline the target in this volume.
2. During patient registration stage, just prior to radiation delivery, acquire a series pairs of N X-rays, X-Rays(i), i=0, . . . , N−1, and N samples of the signals from the LEDs, LEDs(i), over 1 respiratory cycle at the cardiac phase Φ. FIG. 2 shows this scenario with N=11.
3. For each i=0, . . . , N−1, register X-Rays(i) with the CT volume by correlating DRRs with X-rays(I) using a similarity measure or metric. The correlation focuses on registering structures of the heart visible in the DRRs and X-rays such as:
   Any natural landmarks of the heart such as points, lines, surfaces and volumes in, on, and/or around the heart. The silhouette of the heart is one such example. Other examples include parts of the esophagus, the trachea, the bronchial tree, the lungs, the ribs, the diaphragm, the clavicles, the right atrium, the left atrium, the right ventricle, the left ventricle, inferior vena cava, superior vena cava, ascending aorta, descending aorta, pulmonary veins, pulmonary arteries, the heart/lung border and the blood pool. Any artificial landmarks such as one or more fiducials inserted in to the esophagus, the trachea, the bronchial tree, or a catheter placed inside the heart.
4. Optionally, pre-process X-rays, CT volume or DRRs using techniques such as:
   Filtering (thresholding, gradient detection, curvature detection, edge enhancement, image enhancement, spatial frequency-based adaptive processing).
   Segmentation
   Mapping, such as windowing, nonlinear mapping
   Histogram equalization
   Spatial windowing, such as region-of-interest
   Higher order processing, such as connectivity model
   Temporal processing, such as filtering, convolving, differentiation, integration, motion analysis and optical flow.
5. Transform the target location from CT to the coordinate system of the treatment delivery system using the registration step in #3 above. Let the target location in the coordinate system of the treatment delivery system be P(i), i=0, . . . , N−1.
6. Build a correlation model between the target and the LED signals using LEDs(i) as input and P(i), as output. Alternatively build a correlation model between the structures described in #3 above and the LED signals. The location of the target can be computed by adding the offset between the structures and the target to the motion of the structures predicted by the model.
7. Once the correlation model is built, use future samples of LEDs to position and orient the radiation beams.
8. Monitor the validity of the correlation model by acquiring X-ray images intermittently at the cardiac phase, Φ, at any phase of the respiratory cycle.

In Step 2 above both X-ray images and LED signals can be acquired using either prospectively or retrospectively gating. In prospective gating, the ECG waveform may be analyzed by a system module and X-ray images and LED signals can be acquired when the cardiac phase Φ arrives in time. In retrospective gating, the X-ray images, LED signals and ECG samples are continuously acquired and saved with their respective time stamps. Later a separate module compares the time stamps of X-ray images and LED signals to the time stamps of the ECG samples to sort them into the appropriate cardiac phase. Alternatively, if retrospective gating is used, multiple CT volumes, CT(j), j=0, . . . , M−1, at cardiac phases Φ(j) may be acquired in Step 1 and X-rays and LED signals in Step 2 may also be acquired at anyone of the cardiac phases, Φ(j). The registration in Step 3 will then be done by using CT and X-ray images corresponding to the same cardiac phase, Φ(j).

Case 2: With Significant Cardiac Component and with Significant Respiratory Component

The target in the heart muscle has both a respiratory component and a cardiac component. Targets in the ventricles near the valves fall into this category.

### Approach 1:

1. Acquire a series of M CT volumes, CT(j), j=0, . . . , M−1, of the heart over one cardiac cycle with the patient holding his/her breath. Use a high speed CT scanner such as 64-slice Siemens SOMOTOM Definition to acquire CT volumes quickly, e.g. one volume in 83 ms. Contrast agents may be used.
2. FIG. 3 shows a typical EKG waveform with M=10 phases where 10 CT volumes are acquired. Outline the target in each of these M volumes. Alternatively, outline the target in one CT volume and automatically track it over all the CT volumes to generate the targets in other CT volumes.
3. Pick one of the CT phases, Φ, as the reference phase. Acquire a series of pairs of N X-rays, X-rays(i), i=0, . . . , N−1, and N samples of the signals from the LEDs, LED(i), over 1 respiratory cycle at the cardiac phase Φ as in Case 1 (FIG. 2) using prospective or retrospective cardiac gating as before. Build a correlation model between LEDs(i) and X-rays(i) by following steps 3, 4, 5 and 6 in Case 1 and using the CT data from the cardiac phase, Φ.
4. Following this, use the LED signal, LEDs(i) signal to determine the location of the target in the CT volume corresponding to cardiac phase, Φ, assuming the heart does not move due to cardiac motion (similar to Case 1). Then use the EKG signal, EKG(i), to determine the present cardiac phase, and add the offset off the target between the CT volumes of the present cardiac phase and the cardiac phase, φ, to superimpose the cardiac motion component, and thereby to determine the present target position.
5. Monitor the validity of the correlation model by acquiring X-ray images, X-rays(i), intermittently.

### Approach 2:

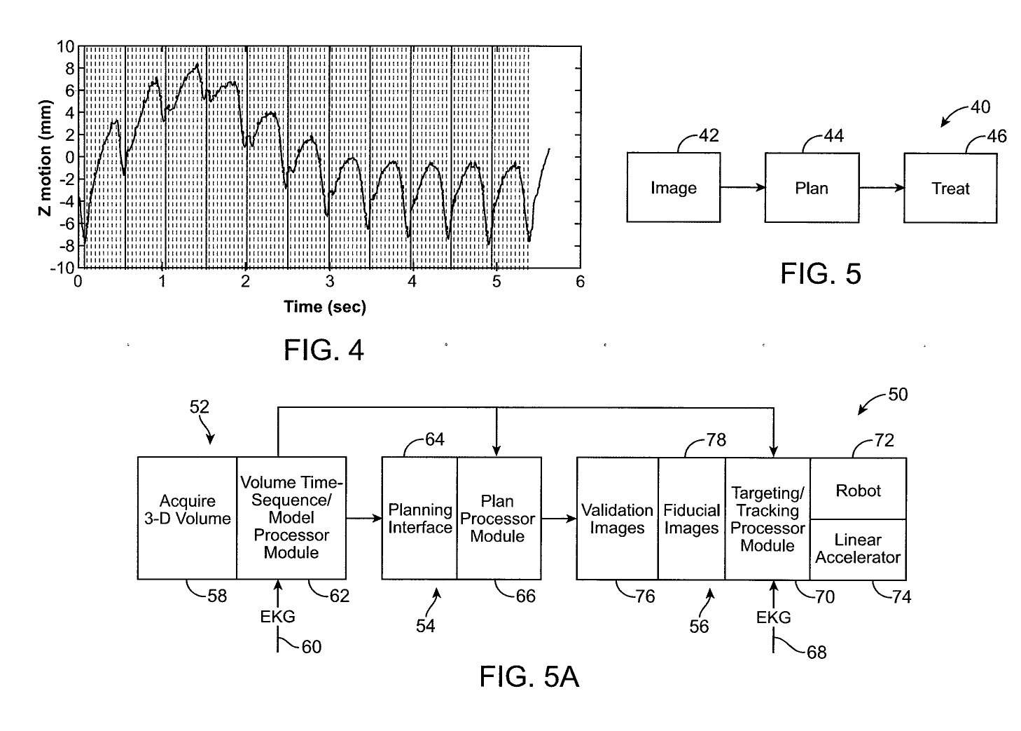

1. Acquire a series of M CT volumes, CT(j), j=0, . . . , M=1, of the heart over one cardiac cycle with the patient holding his/her breath. Use a high speed CT scanner such as 64-slice Siemens SOMOTOM Definition to acquire CT volumes quickly, e.g. one volume in 83 ms. Contrast agents may be used.
2. FIG. 3 shows a typical EKG waveform with M=10 phases where 10 CT volumes are acquired. Outline the target in each of these M volumes. Alternatively, outline the target in one CT volume and automatically track it over all the CT volumes to generate the targets in other CT volumes.
3. During patient registration stage, just prior to radiation delivery, over one respiratory cycle, acquire:
A series pairs of N×M X-rays, X-rays(i,j),
Using the LED signals, bin each X-ray image pair in to one of N respiratory phases and
Using the ECG signals, bin each X-ray image pair in to one of M cardiac phases.
where, i=0, . . . , N−1, j=0, . . . , M−1, i iterates over the phases of a respiratory cycle and j iterates over the phases of a cardiac cycle. The respiratory cycle is divided in to N respiratory phases and each respiratory phase is divided in to M cardiac phases. FIG. 4 shows this scenario, schematically showing M cardiac phases during which a total of M×N X-rays, LED The X-ray acquisition can be prospectively or retrospectively to either or both respiratory and ECG cycles.
   4. For each i=0, . . . , N−1 and j=0, . . . , M−1, register X-rays(i,j) with the CT(j) volume by correlating DRRs with X-rays(i,j). The correlation focuses on registering structures of the heart visible in the DRRs and X-rays such as:
a. Any natural landmarks of the heart such as points, lines, surfaces and volumes in or on the heart. The silhouette of the heart is one such example, and other examples include those discussed above regarding Case 1.
b. Any artificial landmarks such as one or more fiducials inserted in to the esophagus or a catheter placed inside the heart.
   5. Optionally, pre-process X-rays, CT volume or DRRs using techniques such as:
c. Filtering (thresholding, gradient detection, curvature detection, edge enhancement, image enhancement, spatial frequency-based adaptive processing).
d. Segmentation
e. Mapping, such as windowing, nonlinear mapping
f. Histogram equalization
g. Spatial windowing, such as region-of-interest
h. Higher order processing, such as connectivity model
i. Temporal processing, such as filtering, convolution, differentiation, integration, motion analysis and optical flow.
   6. Transform the target location from CT(j) to each of the coordinate system of the treatment delivery system using the registration step in #4 above. Let the target location in the coordinate system of the treatment delivery system be P(i,j).
   7. Build a correlation model between the target and the physiologic cycle data using the respiratory phase (such as the LED signal) and cardiac phase (such as EKG signal) as input and P(i,j) as the output. Alternatively build a correlation model between the structures described in #4 above and the respiratory and cardiac phases. The location of the target can be computed by adding the offset between the structures and the target to the motion of the structures predicted by the model.
   8. Once the correlation model is built, monitor the respiratory and cardiac signals (using LED and EKG data) continuously, determine the respiratory and cardiac phases, predict the target location, P(I,j) and (j) to position and orient the radiation beams.
   9. Monitor the validity of the correlation model by acquiring X-ray images, X-rays(i,j), and the corresponding respiratory and cardiac phases intermittently.

Referring now to FIGS. 5 and 5A, a relatively simple treatment flowchart 40 can represent steps used before and during radiosurgical treatment according to embodiments of the present invention. The internal tissues are imaged 42, typically using a remote imaging modality such as computed tomography (CT), magnetic resonance imaging (MRI), ultrasound imaging, X-ray imaging, optical coherence tomography, a combination of these or other imaging modalities, and/or the like. Note that the tissue structure which will be targeted need not necessarily be visible in the image, so long as sufficiently contrasting surrogate imagable structures are visible in the images to identify the target tissue location. The imaging used in many embodiments will include a time sequence of three dimensional tissue volumes, with the time sequence typically spanning one or more cycles (such as a cardiac or heartbeat cycle, a respiration or breathing cycle, and/or the like).

Based on the images, a plan 44 will be prepared for treatment of the target tissue, with the plan typically comprising a series of radiation beam trajectories which intersect within the target tissue. The radiation dose within the target tissue should be at least sufficient to provide the desired effect (often comprising ablation of tissue, inhibition of contractile pathways within the heart, inhibition of arrhythmogenesis, and/or the like). Radiation dosages outside the target tissues will decrease with a relatively steep gradient so as to inhibit damage to collateral tissues, with radiation dosages in specified sensitive and/or critical tissue structures often being below a desired maximum threshold to avoid deleterious side effects. Embodiments of the invention may employ the 3-D volumes acquired in the imaging step 42 during the planning 44, with exemplary embodiments making use of the motion model represented by the time sequence of 3-D tissue volumes so as to more accurately identify exposure of radiation outside of the target, within sensitive tissue structures, inside the target, and the like. Planned timing of some or all of a series of radiation beams may be established based on the cardiac cycle, the respiration cycle, and/or the like so as to generate the desired dosages within the target tissue, so as to minimize or inhibit radiation exposure to critical structures, and/or to provide desired gradients between the target tissue and collateral or sensitive structures. In some embodiments, the order of the planned radiation beams may be altered and/or the trajectories of the radiation beams may be calculated in response to the motion of the model volume. The plan may also take an electrogram of the heart into consideration.

Once the plan 44 is established, the treatment 46 can be implemented. The treatment will often make use of a processor to direct movement of a robotic structure supporting a radiation beam source, along with registration, validation, and/or tracking modules which enhance accuracy of the treatment. Tracking may employ the motion model developed during imaging 42, and/or may also employ a separate intra-operative motion model. The treatment 46 step and the associated hardware may use a sensor and/or input for physiological wave forms such as the respiration phase, cardiac phase, and the like for use in such tracking.

Referring to the exemplary simplified functional block diagram 50 of FIG. 5A, imaging 52, planning 54, and treatment 56 steps and/or structures are reflected (with slightly more detail) in the structure of the system provided to treat the heart. Imaging 52, planning 54, and treatment 56 structures are employed, with each structure including an associated processor module. The processor modules will typically comprise computer processing hardware and/or software, with the software typically being in the form of tangible media embodying computer-readable instructions or code for implementing one, some, or all of the method steps described herein. Suitable tangible media may comprise a random access memory (RAM), a read-only memory (ROM), a volatile memory, a non-volatile memory, a flash memory, a magnetic recording media (such as a hard disk, a floppy disk, or the like), an optical recording media (such as a compact disk (CD), a digital video disk (DVD), a read-only compact disk, a read/write compact disk, a memory stick, or the like). The various modules described herein may be implemented in a single processor board of a single general purpose computer, or may be run on several different processor boards of multiple proprietary computers, with the code, data, and signals being transmitted between the processor boards using a bus, a network (such as an Ethernet, intranet, or internet), via tangible recording media, using wireless telemetry, or the like. The code may be written as a monolithic software program, but will typically comprise a variety of separate subroutines and/or programs handling differing functions in any of a wide variety of software architectures, data processing arrangements, and the like. Nonetheless, breaking the functionality of the program into separate modules is useful for understanding the capabilities of the various aspects of the invention.

Addressing the imaging block 52 of block diagram 50 in FIG. 5A, a time-sequence of 3-D volumes may be acquired 58 as described above. Corresponding EKG signals 60 may also be received by the model processor module 62, and the processor may optionally use the EKG signals to time the acquisition of the 3-D volumes. In other embodiments, the respiratory signal may also be received by the model processor module 62, and the processor may optionally use the respiratory signal to time the acquisition of the 3D volumes. The series of radiation beams are planned, typically by a surgeon using a user interface 64 (such as a display and keyboard, mouse, or other input device) to communicate with a plan processor module 66. The processor module may make use of the model (including the tissue movements) to determine dosages in the target, collateral, and critical or sensitive tissues.

Once the patient is positioned for treatment relative to the treatment structure 56, an EKG sensor is coupled to the patient to provide EKG signals 68 to the targeting processor module 70. Once again, alternative embodiments may provide respiratory signals. The targeting module configures the robot 72 so as to position and orient the linear accelerator 74 (or other radiation source) toward the target tissue along the desired trajectory for a particular radiation beam from among the series. Once the moving target tissue and the beam trajectory are appropriately aligned, the tracking module 70 may fire the radiation beam by energizing the linear accelerator 74. Hence, the tracking module benefits from the motion model developed during the imaging steps, and the model may optionally be revised using data obtained immediately before and/or during treatment.

Registration and validation of tracking may be provided using X-ray images or the like from a remote image capture system 76, with the exemplary images being provided by a biplanar intermittent X-ray system such as that commercially implemented in the CyberKnife radiosurgical system. Alternatively, a biplanar fluoroscopy X-ray system acquiring images at a high frame rate, for example at a rate of 15 Hz or more, may also be used. Additionally, tracking of respiration-induced movement and the like may be provided using surface image capture devices 78 such as cameras, infrared cameras, or the like to generate signals indicating movement of surface fiducials. Input from the X-ray imaging system 76 and surface image system 78 is also received by the tracking processor module 70.

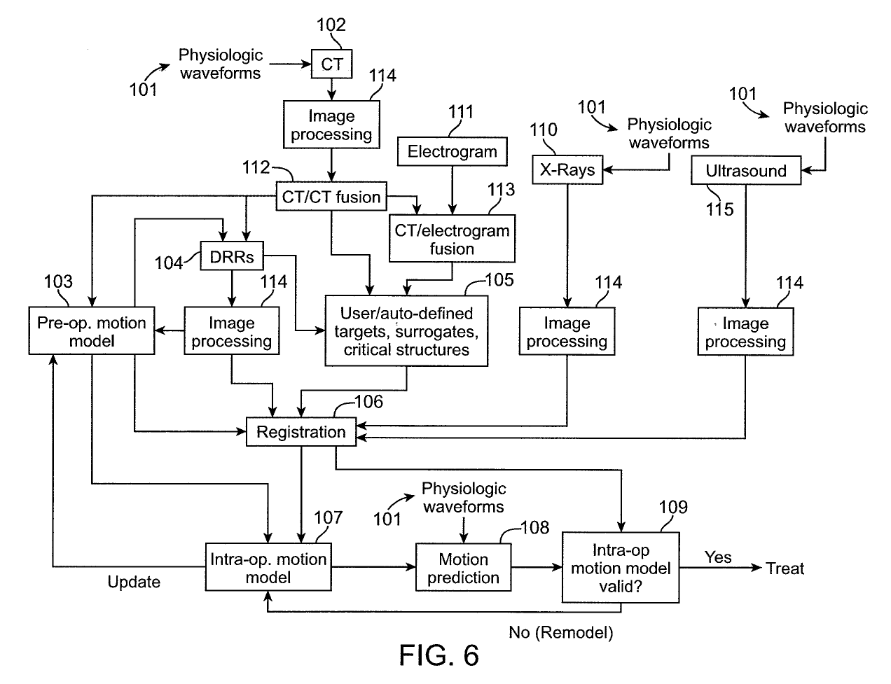

A more comprehensive functional block diagram of an exemplary heart treatment system 100 is schematically illustrated in FIG. 6. System 100 generally registers a series of radiation beams with a target despite motion of the target, often without having to continuously image the moving target. The target will typically comprise an anatomical structure toward which the series of beams converge so as to deposit radiation therein. As the target may be difficult to view in X-ray or other remote imaging modalities, the system may employ surrogate structures, which may be anatomical structures visible in the X-ray near the target which can be aligned and tracked. The surrogate structure may also be an artificial fiducial located near the target. The surrogate structure may alternatively be the same as the target. Alignment generally encompasses the act of registering the CT coordinate system (of a volume acquired from the patient) to the room coordinate system of the treatment system. Tracking encompasses the act of determining the target coordinates in the room coordinate system using, for example, a recent pair of bi-plane X-ray images and the surface images.

The target will generally have motion which includes two components: respiratory motion and cardiac motion. Similarly, the surrogate structure may have two motion components: respiratory motion and cardiac motion.

Referring to the individual components shown in FIG. 6, the physiological wave forms may include ECG signals and respiratory signals (including those derived from images of movement of LEDs or other surface fiducials). CT volumes 102 encompass a variety of different types of CT volumes, and may employ multiple types of CT volumes for a single patient. The CT volumes may be acquired at specific points along the cardiac cycle, respiration cycle, or the like.

Once all the desired CT volumes have been acquired, 2-D and/or 3-D image processing 114 of the acquired images or volumes may be employed. The image processing may include filtering, morphological filtering, mapping, gamma correction, connectivity mapping, distance mapping, order detection, ridge detection, curvature mapping, adaptive filtering, multiscale processing, multi-spectral processing, image enhancement, band pass filtering, unsharp mask filtering, top hat filtering, and/or the like. Many of the acquired volumes may include a series of discrete images at different locations, so that a wide variety of 2-D image filtering and image processing techniques may be employed on the acquired volumes.

Some or all of the acquired CT volumes are fused 112, so that they are registered to a common reference frame. The common reference frame may be based on an anatomical structure such as the spine. Alternatively, deformable registration may be employed, or point-based registration may be used.

An electrogram 111 of a portion or all of the patient's heart may be obtained, and may be fused 113 with the acquired CT volumes. The electrogram may include a voltage map, an activation map, or the like, and may be acquired using commercially available systems such as the Carto™ system commercialized by Biosense Webster (a Johnson & Johnson company). Fusion of the CT volumes with the electrogram can effectively superimpose the electrogram data with the 3-D information in the CT volume and/or the 4-D information in the motion model, allowing (for example) treatment to be directed toward specific anatomical structures based in part on their mapped activation potentials.

DRRs 104 are generated from the CT volumes using any of a variety of techniques, including those described in U.S. Patent Publication No. 2006/0002630. The DRRs will often correspond or approximately correspond in orientation and location to 2-D X-rays 110 obtained by the treatment system while the patient is positioned for treatment. X-rays 110 may be obtained at desired phases of physiological wave forms 101, and may comprise fluoroscopic X-rays or other planar X-ray imaging types, with the X-rays typically being acquired from two or more views simultaneously, such as in the bi-planar X-ray system of the CyberKnife radiosurgical system.

In the planning stage, the system user and/or processor defines targets, surrogates, and critical or sensitive structures using the acquired CT volumes, the DRRs, the electrograms, and/or other available input. A pre-treatment motion model 103 may be generated using the acquired CT volumes, the images of the DRRs, or other two or three dimensional information about the target and surrounding anatomy. The motion model 103 also employs the physiological wave forms 101, and most often the cardiac and/or respiratory phase information associated with each of the acquired 3-D volumes. A parametric motion model may be fitted to the data, or the raw data itself may be used so as to produce a lookup table (where the input is one or more physiological wave forms, and the output is the motion or a quantity derived from the motion such as position, velocity, acceleration, or the like for a given anatomical location in the 3-D space of the model volume or within a 2-D planar space corresponding to the DRR). The pre-treatment motion model 103 may be applied to the CT volume data to generate a new DRR. The DRRs may, for example, have a desired associated cardiac phase, respiration phase, or the like.

Registration 106 encompasses registering the DRRs and the X-rays, with or without use of the pre-treatment motion model. Registration may be a rigid registration or deformable registration, and may comprise registration in 1, 2, 3, 4, 5, or 6 dimensions. Registration could be separable, first performing the registration in a subset of dimensions, followed by registration in another subset of dimensions. Registration may also be a multi-scale registration. Registration 106 may employ multiple disjointed regions of interest (ROI) simultaneously. In exemplary embodiments, registration could be performed using different registration strategies, each fine-tuned to different X-ray views. The results of the registration strategies could depend on the results of other registration strategies.

Intra-operative motion model 107 will often employ the results of the pre-treatment motion model 103, together with the movement identified in the X-rays 110, in ultrasound imaging 115, and the like (often through matching of the surrogates) so as to describe the motion of the target and the sensitive structures with respect to the physiologic wave forms 101. The pre-treatment motion model 103 may be updated based on the information obtained as the system prepares for or implements the series of radiation beams using the intra-treatment motion model 107. Motion is predicted 108 using the intra-operative motion model 107 per the physiologic wave form signals 101, and the intra-operative motion model is validated 109 (typically by checking the predicted position and/or motion of the target or surrogate structures against the actual position and/or motion determined by the registration 106 of the most recent X-ray images 110, ultrasound images 115, and/or the like. If the model does not sufficiently accurately predict the motion and is thus not sufficiently valid, treatment may be interrupted, a new model may be built from scratch and/or the prior intra-operative model may be revised. If the model is within the desired threshold of accuracy, the treatment proceeds.

Referring now to FIGS. 5A and 6, CT volumes may be acquired (reference numerals 58 and 102) using a variety of different approaches. A cardiac gated CT volume may be acquired at a particular phase of the EKG cycle. Two variations of cardiac gated CT may include a held-breath version and a free-breathing version. In the held-breath cardiac gated CT, the patient is holding their breath (typically either at full inspiration or full expiration), so that respiration motion is absent while the data is acquired. In the free breathing cardiac gated CT, the patient is breathing freely. The CT volume may be acquired at a desired point of the respiration cycle. By measuring the respiration wave form, the exact respiratory phase at which the CT volume is acquired can be known (similar to the known cardiac phase at which the CT volume is acquired). In either variation, both the cardiac phase and the respiration cycle phase can be identified for the cardiac gated CT.

A cardiac gated 4-dimensional CT can be generated by acquiring a time series of cardiac gated CT volumes at a series of desired EKG phases. Once again, the 4-D cardiac gated CT can be a held-breath type or a free-breathing type (as described above). Additionally, regarding the free-breathing cardiac gated 4D CT, the resulting series of CT volumes may be acquired at the same EKG phase, typically throughout the respiration cycle. By associating each CT volume with the associated phase of the respiration cycle, the time series CT volumes can be used to model respiratory-induced motion of tissue while minimizing the cardiac motion artifacts.

Yet another type of volume which may be acquired is the respiratory-gated CT volume. Such CT volumes may be acquired at a particular phase of the respiration cycle. The cardiac motion may generally be ignored in this type of CT volume, so that the rapidly moving cardiac structures may be blurry in such CT volumes. In a related respiratory-gated 4-D CT volume, a series of respiratory-gated CT volumes are acquired at a series of respiratory phases.

DRRs may be generated by simulating an X-ray at a desired imaging plane by modeling rays directed through a CT volume. The entire CT dataset need not be used, and thin-slab DRRs may be generated by limiting the mathematical modeling of the effects of the interaction of the X-ray photons with tissue along the rays to the region of the CT volume between any desired start and end point within the CT dataset. Thick-slab DRRs may alternatively model the effects on rays with the start and end points of rays at the limits of the CT dataset. Thin-slab DRRs allow confusing anatomical structures in front of and behind the surrogates (or otherwise outside the region of interest) to be removed or avoided, thus improving registration. Thin-slab DRRs also help allow appreciation of dominant structures visible in the X-rays that correspond to surrogate structures by the user.

Still further improvements in the DRRs may be provided, including the removal of bony anatomy, deformable registration of CT data with X-rays, and the like.

An exemplary patient treatment methodology may clarify the systems and methods described above. In an exemplary treatment, the patient may be treated for atrial flutter although many of the steps to be described may also be applicable to treatment of atrial fibrillation and other arrhythmias. In this embodiment, an anatomical target corresponding to a site of arrhythmogenesis may be chosen for ablation. Such ablation of an anatomic area in the heart can interrupt aberrant pathways or destroy a focus responsible for the arrhythmia. If an electrical map is available outlining abnormal conduction (such as an electrogram using the Carto™ system) the electrical map is correlated to an anatomic site within the heart.

A catheter is placed from a percutaneous venipuncture to the interior of the right atrium under fluoroscopic guidance. In the case of atrial flutter, the catheter may be positioned in, temporarily affixed to, and/or disposed near the ostium of the coronary sinus. Alternatively, the catheter can be placed deep in to the coronary sinus, may engage a cavotricuspid isthmus, and/or may be within a left atrium of the heart, a pulmonary artery outflow tract, a left ventricular outflow tract, a pulmonary vein and/or an ostium of a pulmonary vein. One or more fiducials can also be included in such catheter. The coronary sinus structure is anatomically close (roughly about 1 cm) to the cavotricuspid isthmus, which is often the site of generation of atrial flutter rhythms. The os of the coronary sinus may also move in correlation with the cavotricuspid isthmus. Another catheter may be separately placed via venipuncture and positioned directly on the target cavotricuspid isthmus if desired. Each catheter will have an imagable material near the associated anatomy to be targeted or used as a surrogate structure and may be functioning as a fidicial. The electrodes in ablation catheters may be used as the imagable material and/or a marker. This coronary sinus structure can be accessed with an appropriate catheter tip and moves synchronously with the target in three dimensions, so that knowing the position of such a surrogate fiducial catheter allows one to accurately target the desired anatomical structure. The catheter is visible within the CT volume and X-rays, and can be removed after treatment. Catheters and hence their fiducials can also be temporarily affixed to the cardiac tissue by mechanical means such as using a screw. A cardiac pacing lead is an example.

A CT scan is performed using both cardiac and respiratory-gating so as to obtain a 3-D motion model corresponding to cardiac cycle movement, respiration cycle movement, and/or both. The CT data is fed into the treatment planning module, allowing a library of images to be viewed and the target volume to be identified in three dimensions.

An electrophysiologist and/or cardiologist (for example, the treatment planning physician) may work with a radiation oncologist to generate a treatment plan that deposits radiation with the desired dose at the targeted area (in our example in the cavotricuspid isthmus) so as to inhibit atrial flutter. The radiation dose will result in ablation of the myocardium and will interfere with the abnormal pathway or focus of the arrhythmia. The prescribed dose will typically be in a range from about 15 to about 80 Gy to achieve the desired ablation, and the ablated region may be planned conformably (with consideration of a concentric deposition of dose around an isocenter) or non-conformably (to adjust the dose shape deposited to avoid nearby critical or sensitive structures that the treating physician(s) desires to avoid exposing to excessive radiation). The treatment plan may be reviewed for (among other considerations) the dose, the targeted anatomy, avoidance of critical or sensitive structures near the target, or through which radiation beams should not pass, modification of treatment to the target based on consideration of the motion at the target (based on respiratory and/or cardiac cycle contributions) and/or the like.

The treatment plan is transmitted into the treatment system, and the patient is positioned on the treatment table. Respiratory cycle indicators such as sensors or LEDs can be placed on the chest wall of the patient to provide information (optionally via surface imaging) to the treatment system regarding chest wall motion. The treatment system processor module may predict and/or verify the motion of the target and/or surrogate structures by identifying the respiratory cycle using an intra-treatment model as described above. The patient may also have cutaneous electrocardiogram electrodes placed such that the treating physician and treatment processor module can monitor the cardiac rhythm that the patient is undergoing during treatment.

The treatment takes place by configuring the robot and energizing the radiation source per the series of radiation beams that have been planned. The patient may be monitored via closed circuit TV and/or using sensors such as a heart rate monitors, blood pressure monitors, and other biosensors for any changes during treatment. At the completion of treatment, cutaneous sensors and catheters can be removed. The patient's cardiac rhythm may be monitored remotely via telemetry during a follow-up period.

While the exemplary embodiments have been described in some detail, by way of example and for clarity of understanding, those of skill in the art will recognize that a variety of modification, adaptations, and changes may be employed. Hence, the scope of the present invention should be limited solely by the appending claims.

What is claimed:

1. A method for treating a moving target tissue, the method comprising:
acquiring at least one image of the target tissue;
generating a simulated image from a model volume;
computing a similarity measure between the image(s) and the simulated image;
configuring a robot in response to the similarity measure; and
firing a radiation beam from the configured robot.

2. The method of claim 1, wherein the target tissue comprises a target heart tissue within a heart of a patient, wherein a series of radiation beams are fired from the robot along different trajectories from outside the patient and through intervening tissue toward the target tissue, and further comprising:
generating the model volume before the series of radiation beam by acquiring a time-sequence of volumes and associated cardiac cycle phase measurements, the model volume comprising a model of movement of the target tissue with the cardiac phases, wherein the simulated image has an associated cardiac phase determined using the cardiac phases of the volumes;
wherein each acquired image is acquired during the series of radiation beams, wherein a cardiac phase associated with each acquired image is identified from cardiac signals of a cardiac sensor, and wherein the cardiac phase(s) associated with the simulated image(s) and the cardiac phase of the acquired image are correlated when the similarity measure is computed.

3. The method of claim 2, wherein acquiring each volume used in the model volume comprises imaging a plurality of cross-sectional slices within the heart, wherein the target tissue is sufficiently limited in contrast within the model volume to inhibit modeling of the target tissue movement during the time sequence and/or sufficiently limited to inhibit tracking of target tissue movement in response to the acquired image during the series of radiation beams, and further comprising temporarily introducing at least one imagable material into the blood within the heart so that the material can be absent from the heart after the sequence of radiation beams.

4. The method of claim 3, wherein the imagable material comprises a contrast agent present in the blood within the heart when the time sequence of volumes is acquired using computer tomography.

5. The method of claim 3, wherein the imagable material comprises a catheter advanced through a blood vessel and into the heart so as to provide a temporary fiducial within the heart during acquisition of the images using x-ray imaging.

6. The method of claim 2, wherein the movement model comprises a cardiac cycle movement model and a respiration cycle movement model, wherein the time sequence of volumes used to generate the cardiac cycle movement model are acquired while the patient is holding their breath so as to inhibit respiration-induced movement artifacts, and wherein the respiration cycle movement model is generated using a time sequence of volumes acquired during a respiration cycle extending over a plurality of associated cardiac cycles, the volumes of the respiration cycle movement model acquired at a common cardiac phase during each of the associated cardiac cycles so as to inhibit cardiac cycle-induced movement artifacts in the respiration movement model.

7. The method of claim 2, further comprising planning the series of radiation beams using the model volume.

8. The method of claim 7, wherein the model volume comprises a pre-treatment model, and further comprising:
generating an intra-operative motion model by acquiring a time sequence of images from adjacent the target tissue and a plurality of external fiducials throughout a respiration cycle when the patient is positioned for the series of radiation beams;
imaging the external fiducials during the series of radiation beams;
electrocardiogram monitoring of the cardiac cycle during the series of radiation beams;
predicting motion of the target tissue during the series of radiation beams in response to the imaged external fiducials and the electrocardiogram monitoring; and
verifying the intra-operative motion model by intermittently acquiring images from adjacent the target tissue, the intermittent images being acquired at a rate lower than the respiration rate.

9. The method of claim 2, further comprising obtaining an electrogram of the heart throughout a cardiac cycle, superimposing the electrogram onto the volumes, and planning the series of radiation beams so as to inhibit an arrhythmia of the heart using the superimposed electrogram/volumes by inhibiting a contractile tissue pathway of the heart.

10. The method of claim 2, wherein the heart has an arrhythmia, wherein the radiation beams are directed to the target tissue so as to alleviate the arrhythmia, and further comprising generate the series of radiation beams in response to an arrhythmia type of the arrhythmia.

11. The method of claim 2, wherein the heart has an arrhythmia, wherein the radiation beams are directed to the target tissue so as to alleviate the arrhythmia, and further comprising processing the cardiac signals in response to an arrhythmia type so as to alter the series of radiation beams during the series of radiation beams.

12. The method of claim 11, wherein the arrhythmia type comprises an intermittent arrhythmia and wherein the series of radiation beams are interrupted while the processing of the cardiac signals indicates an acute arrhythmia event.

13. The method of claim 11, wherein the arrhythmia type comprises a chronic atrial fibrillation and wherein the series of radiation beams are interrupted while the processing of the cardiac signals indicates a normal sinus rhythm.

14. A method for treating a moving target tissue of the heart, the method comprising:
acquiring at least one computer tomography (“CT”) volume of the heart;
acquiring at least one X-ray of the heart;
generating a digitally reconstructed radiograph (“DRR”) from the CT volume;
computing a similarity measure between the X-ray and the DRR;
configuring a robot dependent on the similarity measure; and
firing a radiation beam from the configured robot.

15. The method of claim 14, further comprising acquiring a time sequence of CT volumes of the heart and associated electrocardiogram (“ECG”) signals, and configuring the robot in response to movement of the target tissue in the time sequence of CT volumes and in response to ECG signals sensed during firing of the radiation beam.

16. The method of claim 14, wherein the similarity measure is computed using a landmark in the X-ray and the DRR.

17. The method of claim 16, wherein the landmark comprises a cardiac landmark selected from the group comprising a cardiac silhouette, an esophagus, a trachea, a bronchial tree, a lung, a rib, a diaphragm, a clavicles, a right atrium, a left atrium, a right ventricle, a left ventricle, an inferior vena cava, a superior vena cava, an ascending aorta, a descending aorta, a pulmonary vein, a pulmonary artery, a heart/lung border and a blood pool.

18. The method of claim 16, wherein the landmark comprises a catheter extending into, engaging, and/or affixed to, so as to move with, a coronary sinus, a cavotricuspid isthmus, a left atrium of the heart, a pulmonary artery outflow tract, a left ventricular outflow tract, a pulmonary vein and/or the ostium of a pulmonary vein.

19. A system for treating a moving target tissue, the system comprising:
an image acquisition system for acquiring at least one image of the target tissue;
a processor coupled to the image acquisition system, the processor configured for:
generating a simulated image from a model volume, the model volume including a motion model of the target tissue;
computing a similarity measure between the image and the simulated image; and
determining a configuration in response to the similarity measure;
a robot coupled to the processor for implementing the configuration; and
a radiation beam source supported by the robot.

20. The system of claim 19, the target tissue comprising a target heart tissue, further comprising a cardiac cycle sensor coupled to the processor, the processor associating a phase of the cardiac cycle with the acquired image per signals from the sensor, and wherein the configuration is determined in response to the cardiac cycle associated with the image.

21. The system of claim 20, wherein the processor computes a series of radiation beams having different trajectories from outside the patient to the target tissue, and further comprising:
a 3-D imaging system coupled to the processor so as to transmit a time-sequence of volumes thereto, the processor generating a movement model volume from the time-sequence of volumes and associated cardiac phase data, the movement model indicating movement of the target tissue with the cardiac phases, wherein the simulated image generated by the processor has an associated cardiac phase determined using the cardiac phases and/or respiratory phases of the volumes, and wherein the cardiac phase(s) associated with the simulated image(s) and the acquired images correlate when the similarity measure is computed.

22. The system of claim 21, wherein the 3-D imaging system comprises a computer tomography (“CT”) system that acquires each volume of the time-sequence as a plurality of cross-sectional slices within the heart, wherein the target tissue is sufficiently limited in contrast to inhibit modeling of the target tissue movement during the time sequence and/or sufficiently limited to inhibit tracking of target tissue movement in response to the acquired image during the series of radiation beams, and further comprising at least one imagable material temporarily introducing into the blood within the heart so as to safely enhance modeling of target tissue movement and/or target tissue tracking.

23. The system of claim 22, wherein the imagable material comprises a contrast agent releasable into the blood within the heart.

24. The system of claim 22, wherein the imagable material comprises a coronary catheter advanceable through a blood vessel and into the heart, the catheter temporarily affixable to the heart so as to provide a temporary fiducial within the heart.

25. The system of claim 21, wherein the processor is configured to generate the model volume, the model volume comprising a cardiac cycle movement model and a respiration cycle movement model, the cardiac cycle movement model comprising a time sequence of volumes generated while inhibiting respiration-induced movement artifacts, the respiration cycle movement model generated using a time sequence of volumes acquired during a respiration cycle extending over a plurality of associated cardiac cycles, the volumes of the respiration cycle movement model acquired in response signals indicating a common cardiac phase during each of the associated cardiac cycles so as to inhibit cardiac cycle-induced movement artifacts.

26. The system of claim 21, wherein the processor comprises a beam planning module having an interface configured for planning the sequence of radiation beams using the model volume, wherein the model volume comprises a pre-treatment model.

27. The system of claim 26, further comprising a plurality of fiducials adapted to be supported on an external surface of the patient and a surface imaging system coupled to the processor, the processor further comprising a module configured for generating an intra-operative motion model using a time sequence of images from adjacent the target tissue and images of the external fiducials throughout a respiration cycle.

28. The system of claim 26, wherein the processor monitors the cardiac cycle during the sequence of radiation beams using the intra-operative model module to predict motion of the target tissue in response to electrocardiogram signals and the imaged external fiducials and the electrocardiogram monitoring, the processor verifying the intra-operative motion model using intermittent internal images from adjacent the target tissue.

29. The system of claim 19, further comprising an electrogram measurement system coupled to the processor, the processor superimposing the electrogram onto the model volume and planning a series of radiation beams so as to inhibit an arrhythmia of the heart using the superimposed electrogram/volume by inhibiting a contractile tissues pathway of the heart.

30. The system of claim 19, wherein the heart has an arrhythmia, wherein a series of radiation beams are directed to the target tissue so as to alleviate the arrhythmia, and wherein the processor determines the configuration of the robot so as to generate the series of radiation beams in response to an arrhythmia type of the arrhythmia.

31. The system of claim 19, wherein the heart has an arrhythmia, wherein a series of radiation beams are directed to the target tissue so as to alleviate the arrhythmia, and wherein the processor is configured to alter the series of radiation beams during the series of radiation beams in response to an arrhythmia type signal.

32. The system of claim 31, further comprising a cardiac cycle sensor coupled to the processor, wherein the arrhythmia type signal corresponds to an intermittent arrhythmia and wherein the processor is configured to interrupt the series of radiation beams when cardiac signals from the sensor indicates an acute arrhythmia event.

33. The system of claim 31, further comprising a cardiac cycle sensor coupled to the processor, wherein the arrhythmia type comprises a chronic atrial fibrillation and wherein the processor interrupts the series of radiation beams when cardiac signals from the sensor indicates a normal sinus rhythm.

34. A system for treating a moving target tissue of the heart, the method comprising:
a processor;
a computer tomography (“CT”) system coupled to the processor so as to transmit an acquired volume of the heart thereto;
an X-ray system coupled to the processor so as to transmit an acquired image of the heart thereto;
a robot coupled to the processor; and
a radiation source supported by the processor;
the processor having a DRR module generating a digitally reconstructed radiograph (“DRR”) from the CT volume, a similarity module generating a similarity measure between the X-ray and the DRR, the processor configuring the robot dependent on the similarity measure, and firing a series of the radiation beams from the radiation source so as to treat the moving tissue.

35. The system of claim 34, further comprising at least one electrocardiogram (“ECG”) sensor coupled to the processor, wherein the processor stores a time sequence of CT volumes of the heart and associated cardiac phase data based on signals from the electrocardiogram (“ECG”) sensor, and configures the robot in response to movement of the target tissue in the time sequence of CT volumes and in response to ECG signals sensed during the series of radiation beams.
